(19) United States
(12) Patent Application Publication
(10) Pub. No.: US 2026/0363589 A1
(43) Pub. Date: Nov. 19, 2026

(54) BLOOD COLLECTION EJECTION ACTUATING MECHANISM AND LANCET USING THE SAME

(71) Applicant: STERILANCE MEDICAL (SUZHOU) INC., Suzhou City, Jiangsu Province (CN)

(72) Inventors: Guoping SHI, Suzhou City (CN); Anthony Scott HORSTMAN, Suzhou City (CN)

(73) Assignee: STERILANCE MEDICAL (SUZHOU) INC., Suzhou City, Jiangsu Province (CN)

(21) Appl. No.: 19/515,321

(22) PCT Filed: Aug. 5, 2024
(86) PCT No.: PCT/CN2024/109818

§ 371 Date: Feb. 27, 2026

(30) Foreign Application Priority Data
Aug. 31, 2023 (CN) 202311112494.8

(51) Int. Cl. A61B 5/151

(52) U.S. Cl. CPC A61B 5/15126

(57) ABSTRACT

The invention discloses a blood collection ejection actuating mechanism and a lancet using the same. The blood collection ejection actuating mechanism comprises an integrated ejection mechanism component, and an ejection channel, an energy storage channel and an emission channel which are arranged on a housing for the assembly and movement of the integrated ejection mechanism component; the integrated ejection mechanism component comprises an ejection key, a traction elastic arm, a leaf spring and a base body which are integrally and continuously arranged in sequence; a first motion control part is arranged between the traction elastic arm and the leaf spring; a second motion control part and a third motion control part are arranged on the base body. According to the invention, the components for providing ejection actuating for the emission and lancing of the cutting part of the lancet are designed as an integrated ejection mechanism component, and the ejection channel, the energy storage channel and the emission channel are correspondingly arranged on the housing, so that assembly and corresponding motion linkage can be realized, and thus the emission and lancing of the cutting part of the lancet can be completed by using only one integrated ejection mechanism component.

### TECHNICAL FIELD

The present disclosure relates to the technical field of blood collection instruments, and in particular to a blood collection ejection actuating mechanism and a lancet using the same. The lancet adopts a lancing (cutting) blood collection mode, which is mainly applicable to heel blood collection of infants and young children, and can also be applied to blood collection at other parts of the human body.

### BACKGROUND

Lancets, as medical human blood collection devices, are widely used in various medical institutions. Lancets are divided into two types according to the blood collection mode: puncture type and lancing (cutting) type. The puncture type acts on the blood collection site by needle pricking, which is mainly applicable to finger blood collection of adults, while the lancing type acts on the blood collection site by blade lancing. Since the lancing mode causes less pain and can obtain a sufficient blood collection volume, it is particularly suitable for heel blood collection of infants and young children, especially newborn babies.

Chinese Patent CN111449660B discloses an invention patent entitled “lancet”, which provides a technical solution for a lancet. Although this design has reduced the number of parts compared with the existing heel lancets, the applicant finds that its ejection actuating system is composed of three components: a trigger body 14, a cutter holder 30 and an elastic component 40. The design of this ejection actuating system not only involves a large number of parts and is difficult to assemble, but also the elastic component 40 is in a continuous stress state in the initial state, which is prone to failure before use, thus leading to product malfunction.

In view of the above, how to solve the problems of the existing lancets, such as numerous parts, difficult assembly, and easy failure of elastic components before use due to continuous stress in the initial state, has become the technical subject to be researched and solved by the present disclosure.

### SUMMARY OF THE INVENTION

The objective of the present disclosure is to provide a blood collection ejection actuating mechanism and a lancet using the same.

To achieve the above objective, the technical solution adopted by the present disclosure is: the present disclosure provides a blood collection ejection actuating mechanism for providing ejection actuating for the emission and lancing of a cutting part of a lancet, and the lancet comprises a housing with a lancing opening for the cutting part to extend out. The innovation of the present disclosure is as follows:

The blood collection ejection actuating mechanism comprises an integrated ejection mechanism component, and an ejection channel, an energy storage channel and an emission channel which are arranged on the housing for the assembly and movement of the integrated ejection mechanism component;

The integrated ejection mechanism component includes an ejection key, a traction elastic arm, a leaf spring and a base body which are integrally and continuously arranged in sequence; a first motion control part is arranged between the traction elastic arm and the leaf spring; a second motion control part and a third motion control part are arranged on the base body;

The ejection key is provided with a guide part assembled on the ejection channel, and the ejection key is a component that bears an external pressing force and/or a push-pull force, then moves along the ejection channel and pushes the first motion control part to move; a driving surface is arranged on the ejection key;

The first motion control part is assembled on the energy storage channel, and a driven part is arranged on the first motion control part or at the position between the traction elastic arm and the leaf spring corresponding to the driving surface; the first motion control part is a component that bears the acting force of the ejection key, then moves along the energy storage channel and drives the traction elastic arm and the leaf spring to store energy, and when the first motion control part crosses and disengages from the energy storage channel, the first motion control part drives the base body to move under the traction force of the traction elastic arm and the reaction force of the energy storage channel;

The second motion control part and the third motion control part are assembled on the emission channel, and the base body is a component connected with the cutting part; when the ejection key is not subjected to an external acting force, the second motion control part and the third motion control part are located in the emission channel; when the first motion control part moves along the energy storage channel, the movement and rotation tendency of the base body under the elastic action of the leaf spring is blocked by the second motion control part and the third motion control part abutting against the emission channel; when the first motion control part crosses and disengages from the energy storage channel, the second motion control part and the third motion control part move along the emission channel toward the lancing opening driven by the base body; after the second motion control part crosses and disengages from the emission channel, the base body moves to one lower side relative to the lancing opening under the torsion force of the leaf spring and the inertial acting force along the emission channel in the early stage, and accordingly the base body drives the cutting part to perform lancing toward the other lower side.

The relevant contents of the present disclosure are explained as follows:
1. In the above technical solution of the present disclosure, in-depth research and design are carried out aiming at the problems of the existing lancets, such as numerous parts, difficult assembly, and easy failure of elastic components before use due to continuous stress in the initial state. The core improvement is that the components for providing ejection actuating for the emission and lancing of the cutting part of the lancet are designed as an integrated ejection mechanism component, and the ejection channel, the energy storage channel and the emission channel are correspondingly arranged on the housing, so that the integrated ejection mechanism component can be assembled and realize corresponding motion linkage, and the whole blood collection ejection actuating mechanism can be sequentially converted into a pre-emission state, an initial emission state, a late emission state and a final emission state in order. Thus, the emission and lancing of the cutting part of the lancet can be completed by using only one integrated ejection mechanism component, and the lancing function is completely the same as that of the existing lancets composed of multiple parts. The ejection key can be pushed to apply force smoothly without jamming; in the pre-emission state, the integrated ejection mechanism component is placed in the housing in a natural unstressed state, and the leaf spring of the integrated ejection mechanism component, which provides elastic acting force for the rotation of the base body, is not stressed, which can well avoid the possibility of product failure before use, and ensure that sufficient and rapid lancing force can be provided for the lancing of the cutting part in the final emission state, so as to guarantee the blood collection volume and reduce pain; when the first motion control part bears the acting force of the ejection key, moves along the energy storage channel and drives the traction elastic arm and the leaf spring to store energy, the movement and rotation tendency of the base body under the elastic action of the leaf spring is blocked first, so that the traction elastic arm and the leaf spring can generate sufficient deformation to store enough elastic acting force; and when the first motion control part crosses and disengages from the energy storage channel, the first motion control part drives the base body to move under the traction force of the traction elastic arm and the reaction force of the energy storage channel, at this time, the traction elastic arm can provide sufficient elastic acting force for the movement of the base body, so that the base body can move rapidly along the emission channel toward the lancing opening with sufficient emission actuating and high emission speed; after the second motion control part on the base body is driven by the rapid traction of the traction elastic arm to cross and disengage from the emission channel, the base body immediately rotates under the action of the leaf spring after the rotation restriction is released, and the leaf spring which has stored sufficient elastic acting force in the early stage can drive the cutting part on the base body to perform rapid rotary lancing with large and high-speed lancing force, thus ensuring the blood collection volume and reducing pain.
2. In the above technical solution, the blood collection ejection actuating mechanism is configured to be sequentially converted into a pre-emission state, an initial emission state, a late emission state and a final emission state in order;

In the pre-emission state, the integrated ejection mechanism component is placed in the housing in a natural unstressed state, the ejection key is mounted on the ejection channel, the first motion control part is located at the placement end of the energy storage channel, and the second motion control part and the third motion control part are located in the emission channel and close to the two sides of the inner wall of the emission channel respectively;

When converting from the pre-emission state to the initial emission state, the ejection key pushes the first motion control part to move along the energy storage channel under an external force, the traction elastic arm and the leaf spring move and store energy synchronously, and the movement and rotation tendency of the base body under the elastic action of the leaf spring is blocked by the two sides of the inner wall of the emission channel;

When converting from the initial emission state to the late emission state, the ejection key continues to push the first motion control part to move along the energy storage channel under the external force, the traction elastic arm and the leaf spring continue to move and store energy synchronously, wherein the first motion control part moves to the critical end of the energy storage channel and is in a critical to-be-disengaged state;

When converting from the late emission state to the final emission state, the ejection key continues to push the first motion control part under the external force to make the first motion control part cross the critical end of the energy storage channel, the first motion control part drives the base body to move along the emission channel toward the lancing opening under the traction force of the traction elastic arm and the reaction force of the energy storage channel, the second motion control part on the base body disengages from the emission channel to release the restriction on the rotation tendency of the base body, and the base body and the cutting part on the base body perform rotary lancing at the lancing opening under the torsion force of the leaf spring.

The orderly conversion of the above various states enables the lancet to realize easy, smooth and effective lancing blood collection, ensures that the leaf spring in the integrated ejection mechanism component will not fail and can provide sufficient elastic acting force, thus guaranteeing the normal operation of blood collection.
3. In the above technical solution, the first motion control part, the second motion control part and the third motion control part are all raised cylindrical bodies, and the ejection channel, the energy storage channel and the emission channel are all grooves or spaces arranged on the housing. This design makes the structural design of the integrated ejection mechanism component, the movement of each component and the realization of the elastic structures of the traction elastic arm and the leaf spring more scientific and reasonable, and makes the whole pushing and emission process of the lancet smoother without jamming.
4. In the above technical solution, a first retaining wall and a second retaining wall are arranged on the housing, the space between the first retaining wall and the second retaining wall forms the emission channel, a disengagement end is arranged on the first retaining wall at a position close to the lancing opening, and an abutment end is arranged on the second retaining wall at a position close to the lancing opening;

In the pre-emission state, the second motion control part is located in the emission channel and close to the first retaining wall, and the third motion control part is located in the emission channel and close to the second retaining wall;

When converting from the pre-emission state to the initial emission state, the movement and rotation tendency of the base body under the elastic action of the leaf spring is blocked by the first retaining wall and the second retaining wall;

When converting from the late emission state to the final emission state, the second motion control part on the base body moves and disengages from the first retaining wall to release the restriction on the rotation tendency of the base body, and the third motion control part moves to the abutment end and is rotatably connected with the abutment end in a matching manner.
5. In the above technical solution, with the end face of the housing where the lancing opening is located as a horizontal reference plane, the horizontal height of one of the second motion control part and the third motion control part is higher than that of the other, thus forming a height difference between the second motion control part and the third motion control part. When converting from the pre-emission state to the initial emission state, this height difference can make the two parts better blocked by the emission channel (the first retaining wall and the second retaining wall) without premature disengagement; and when converting from the late emission state to the final emission state, when one part is released from the restriction of the emission channel, it can better rotate around the other part under the action of the leaf spring with a sufficiently good lancing angle and speed.
6. In the above technical solution, a driving surface is arranged on the ejection key, and a driven part is arranged on the first motion control part or at the position between the traction elastic arm and the leaf spring corresponding to the driving surface; when the ejection key bears an external acting force, the driving surface contacts and acts on the driven part, so that the ejection key drives and pushes the first motion control part to move.
7. In the above technical solution, the driving surface is arranged to be an inclined surface at least at the part in contact with the driven part, and the driven part is arranged to be a curved surface at least at the part in contact with the driving surface, so that the contact part of the driving surface and the driven part on the cross section is a point contact. Thus, when pushing the ejection key to drive the first motion control part to move, especially when the energy storage channel is not a straight channel, the driving surface and the driven part can always keep point contact when the driven part rotates at a certain angle, thereby reducing friction force, making the pushing process smoother, reducing the force applied to push the ejection key and avoiding jamming.
8. In the above technical solution, the traction elastic arm is curved, a curved transition part is arranged on the traction elastic arm at the connection part with the first motion control part, and the traction elastic arm and the transition part are curved in different directions. Specifically, the main body of the traction elastic arm and the transition part are reverse circular arcs, so that when pushing the ejection key and the first motion control part pushes the traction elastic arm and the leaf spring to store energy, the resistance generated after the compression of the traction elastic arm will diverge and not concentrate on a single point; and when the first motion control part disengages from the energy storage channel so that the traction elastic arm provides traction force for the movement of the base body toward the lancing opening, the tension of the traction elastic arm stretches uniformly without being too large or too small, resulting in a better hand feeling.
9. In the above technical solution, the emission channel is obliquely arranged toward the lancing opening, specifically inclined in the general direction toward the lancing opening, so that the base body can move obliquely downward better under the traction of the traction elastic arm, and the process of the disengagement of the second motion control part and the abutment of the third motion control part to the abutment end can be smoother.
10. In the above technical solution:

The ejection key is provided with an ejection part for bearing an external pressing force and/or a push-pull force;

An ejection opening is arranged on the housing at a position corresponding to the ejection part;

When the ejection part is located at the head of the ejection key, the ejection opening is arranged on the peripheral side of the housing;

When the ejection part is located on the two sides of the ejection key, the ejection opening is arranged on the two sides of the housing;

When the ejection part is located at both the head and the two sides of the ejection key, the ejection opening is arranged on both the peripheral side and the two sides of the housing.
11. In the above technical solution, the energy storage channel is curved, and the bending direction of the energy storage channel is set toward the lancing opening, so that the middle position of the energy storage channel is higher than the two ends. The first motion control part is first pushed obliquely upward from the placement end of the energy storage channel to the middle, and then pushed obliquely downward from the middle to the critical end, which makes the energy storage and release process of the traction elastic arm and the leaf spring more reasonable and the operation hand feeling better.
12. In the above technical solution, the housing comprises an upper shell and a lower shell, and the ejection channel, the energy storage channel and the emission channel are arranged on either or both of the upper shell and the lower shell.
13. In the present disclosure, the opening direction of the lancing opening is the extending direction of the cutting part.
14. In addition, the term “and/or” in the present application means including three parallel schemes. Taking “A and/or B” as an example, it includes scheme A, scheme B, or a scheme where both A and B are satisfied.

Due to the application of the above scheme, the present disclosure has the following advantages and effects compared with the prior art:
1. In the scheme of the present disclosure, in the pre-emission state, the integrated ejection mechanism component is placed in the housing in a natural unstressed state, and the leaf spring of the integrated ejection mechanism component, which provides elastic acting force for the rotation of the base body, is not stressed, which can well avoid the possibility of product failure before use, and ensure that sufficient and rapid lancing force can be provided for the lancing of the cutting part in the final emission state, so as to guarantee the blood collection volume and reduce pain.
2. In the scheme of the present disclosure, when the first motion control part bears the acting force of the ejection key, moves along the energy storage channel and drives the traction elastic arm and the leaf spring to store energy, the movement and rotation tendency of the base body under the elastic action of the leaf spring is blocked first, so that the traction elastic arm and the leaf spring can generate sufficient deformation to store enough elastic acting force; and when the first motion control part crosses and disengages from the energy storage channel, the first motion control part drives the base body to move under the traction force of the traction elastic arm and the reaction force of the energy storage channel, at this time, the traction elastic arm can provide sufficient elastic acting force for the movement of the base body, so that the base body can move rapidly along the emission channel toward the lancing opening with sufficient emission actuating and high emission speed.
3. In the scheme of the present disclosure, after the second motion control part on the base body is driven by the rapid traction of the traction elastic arm to cross and disengage from the emission channel, the base body immediately rotates under the action of the leaf spring after the rotation restriction is released, and the leaf spring which has stored sufficient elastic acting force in the early stage can drive the cutting part on the base body to perform rapid rotary lancing with large and high-speed lancing force, thus ensuring a sufficient blood collection volume and reducing pain.
4. In the scheme of the present disclosure, in-depth research and design are carried out aiming at the problems of the existing Lancets, such as numerous parts, difficult assembly, and easy failure of elastic components before use due to continuous stress in the initial state. The core improvement is that the components for providing ejection actuating for the emission and lancing of the cutting part of the lancet are designed as an integrated ejection mechanism component, and the ejection channel, the energy storage channel and the emission channel are correspondingly arranged on the housing, so that the integrated ejection mechanism component can be assembled and realize corresponding motion linkage, and the whole blood collection ejection actuating mechanism can be sequentially converted into a pre-emission state, an initial emission state, a late emission state and a final emission state in order. Thus, the emission and lancing of the cutting part of the lancet can be completed by using only one integrated ejection mechanism component, and the lancing function is completely the same as that of the existing lancets composed of multiple parts. The ejection key can be pushed to apply force smoothly without jamming, and the leaf spring will not fail. Therefore, the present disclosure provides a novel and reasonable structural design with a unique and ingenious technical conception, prominent substantive features and remarkable progress.

### BRIEF DESCRIPTION OF THE DRAWINGS

FIG. 1 is a perspective view of the overall structure of an embodiment of the present disclosure;

FIG. 2 is an exploded schematic view of an embodiment of the present disclosure;

FIG. 3 is a perspective view of an integrated ejection mechanism component in an embodiment of the present disclosure;

FIG. 4 is a front view of an integrated ejection mechanism component in an embodiment of the present disclosure;

FIG. 5 is a schematic structural view of an embodiment of the present disclosure in a pre-emission state;

FIG. 6 is a schematic structural view of an embodiment of the present disclosure in an initial emission state;

FIG. 7 is a schematic structural view of an embodiment of the present disclosure in a late emission state;

FIG. 8 is a schematic structural view of an embodiment of the present disclosure in a final emission state;

FIG. 9 is a schematic view of driving a cutting part to perform rotary lancing in the final emission state according to an embodiment of the present disclosure;

FIG. 10 is a schematic view of assembling a cutting part on the base body of the ejection leaf spring needle holder in an embodiment of the present disclosure;

FIG. 11 is a schematic structural view of a housing in an embodiment of the present disclosure.

The components in the above figures are indicated as follows:
1—Housing; 101—Lancing opening; 102—Ejection opening;
11—Ejection channel;
12—Energy storage channel; 121—Placement end; 122—Critical end;
13—Emission channel; 131—First retaining wall; 1311—Disengagement end; 132—Second retaining wall; 1321—Abutment end;
2—Integrated ejection mechanism component;
21—Ejection key; 210—Driving surface; 2101—Inclined surface; 211—Guide part; 212—Ejection part;
22—Traction elastic arm; 221—Transition part;
23—Leaf spring;
24—Base body; 241—Second motion control part; 242—Third motion control part;

125—First motion control part; 251—Driven part; 2511—Curved surface;
9—Cutting part.

### EMBODIMENTS

The present disclosure will be further described below with reference to the accompanying drawings and embodiments:

Embodiment: The present application will be clearly explained below with the drawings and detailed descriptions. After understanding the embodiments of the present application, those skilled in the art can make changes and modifications by virtue of the techniques taught in the present application without departing from the spirit and scope of the present application.

The terms used herein are only for describing specific embodiments and are not intended to limit the present application. Singular forms such as “a”, “an”, “the” and “this” as used herein also include plural forms.

The terms “first”, “second” and the like used herein do not specifically mean an order or sequence, nor are they used to limit the present application, but only to distinguish components or operations described with the same technical terms.

The terms “connection” or “positioning” used herein may refer to direct physical contact between two or more components or devices, or indirect physical contact between them, and may also refer to mutual operation or movement between two or more components or devices.

The terms “comprise”, “include”, “have” and the like used herein are all open-ended terms, meaning including but not limited to.

Unless otherwise specified, the terms used herein usually have their ordinary meanings in the art, in the content of the present application and in specific contexts. Some terms used to describe the present application will be discussed below or elsewhere in this specification to provide those skilled in the art with additional guidance on the description of the present application.

The directional terms such as “front”, “rear”, “upper”, “lower” used herein are only used to explain the positional relationship between various structures in the present application, and are not used to limit the protection scheme of the present application and the specific directions in actual implementation.

The present disclosure is designed with ingenious improvements aiming at the problems of the existing Lancets, such as numerous parts, difficult assembly, and easy failure of elastic components before use due to continuous stress in the initial state. The core improvement is that the components for providing ejection actuating for the emission and lancing of the cutting part 9 of the lancet are designed as an integrated ejection mechanism component 2, and the ejection channel 11, the energy storage channel 12 and the emission channel 13 are correspondingly arranged on the housing 1, so that the integrated ejection mechanism component 2 can be assembled and realize corresponding motion linkage, and the whole blood collection ejection actuating mechanism can be sequentially converted into a pre-emission state, an initial emission state, a late emission state and a final emission state in order. This design also allows the integrated ejection mechanism component 2 to be placed in the housing 1 in a natural unstressed state, thereby avoiding the deformation and failure of the leaf spring.

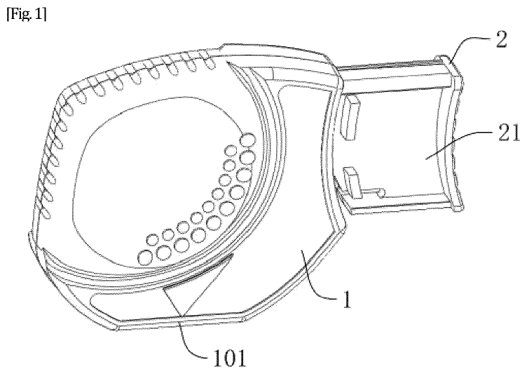

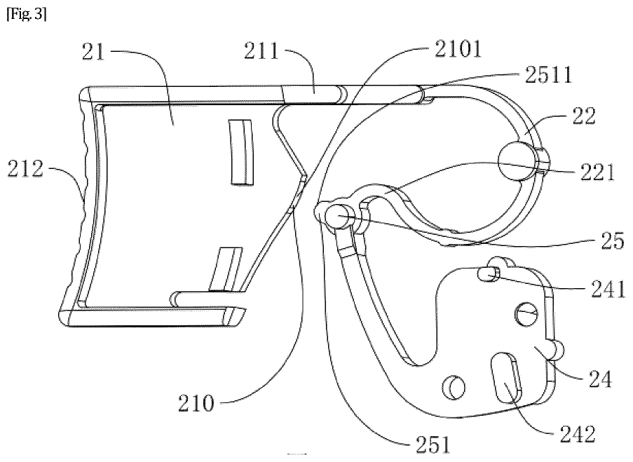

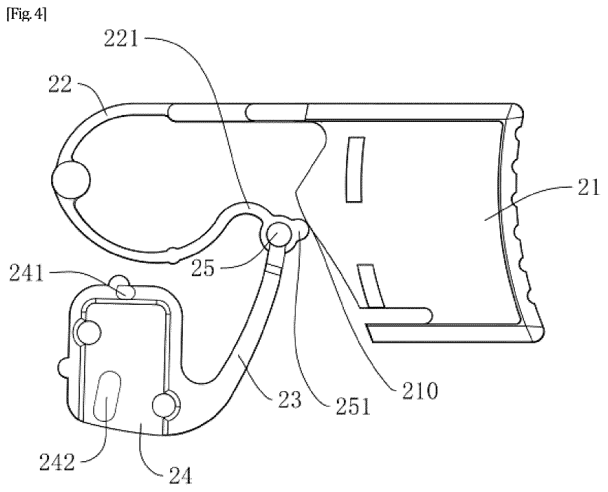

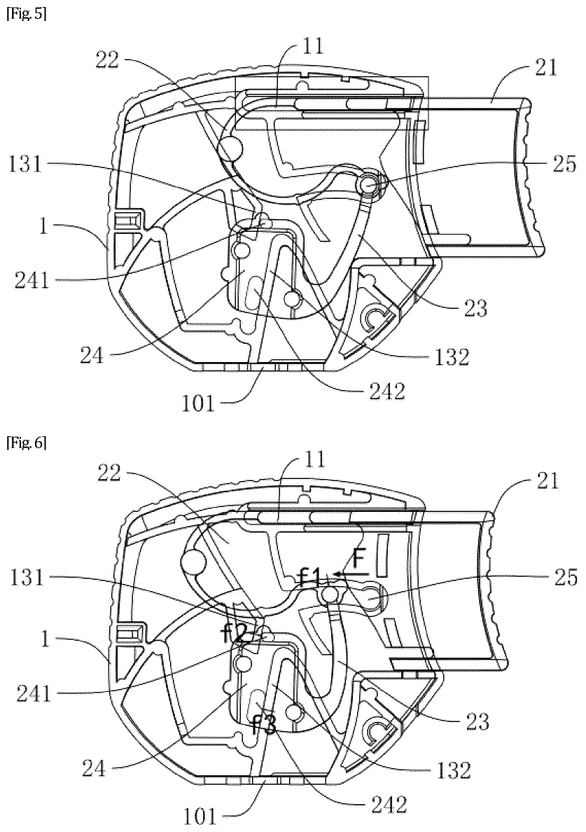

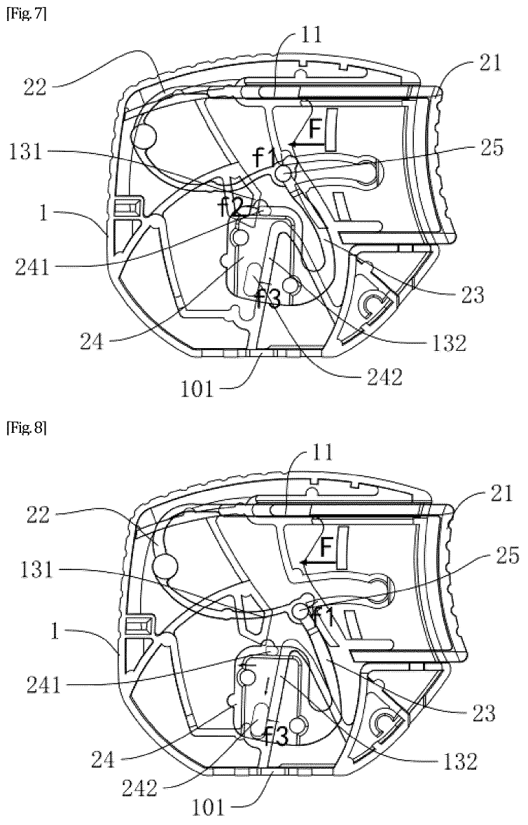

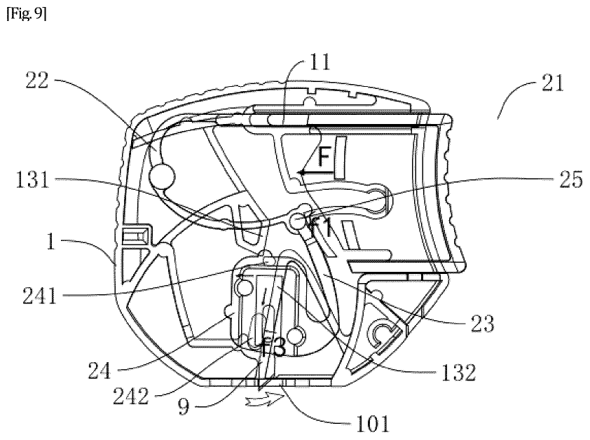

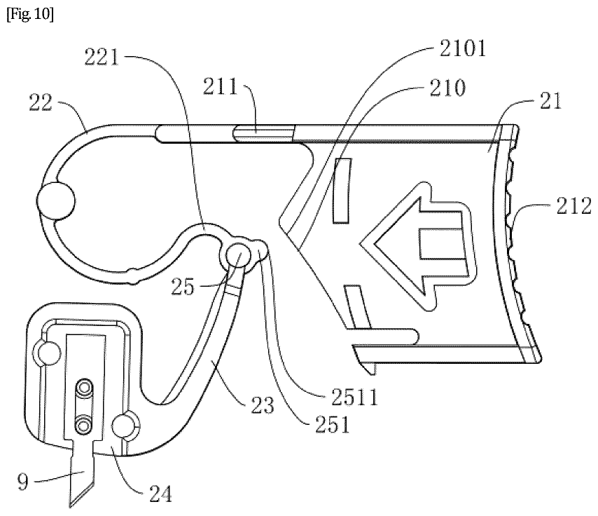

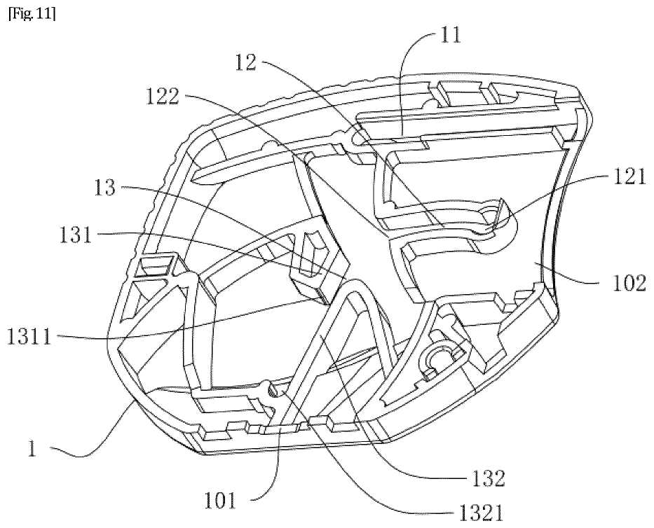

Embodiment 1: Referring to FIGS. 1 to 4, Embodiment 1 of the present disclosure provides a blood collection ejection actuating mechanism for providing ejection actuating for the emission and lancing of a cutting part 9 of a lancet, wherein the lancet comprises a housing 1 with a lancing opening 101 for the cutting part 9 to extend out.

In Embodiment 1 of the present disclosure, the blood collection ejection actuating mechanism comprises an integrated ejection mechanism component 2, and an ejection channel 11, an energy storage channel 12 and an emission channel 13 which are arranged on the housing 1 for the assembly and movement of the integrated ejection mechanism component 2;

The integrated ejection mechanism component 2 includes an ejection key 21, a traction elastic arm 22, a leaf spring 23 and a base body 24 which are integrally and continuously arranged in sequence; a first motion control part 25 is arranged between the traction elastic arm 22 and the leaf spring 23; a second motion control part 241 and a third motion control part 242 are arranged on the base body 24;

The ejection key 21 is provided with a guide part 211 assembled on the ejection channel 11, and the ejection key 21 is a component that bears an external pressing force and/or a push-pull force, then moves along the ejection channel 11 and pushes the first motion control part 25 to move; a driving surface 210 is arranged on the ejection key 21;

The first motion control part 25 is assembled on the energy storage channel 12, and a driven part 251 is arranged on the first motion control part 25 or at the position between the traction elastic arm 22 and the leaf spring 23 corresponding to the driving surface 210; the first motion control part 25 is a component that bears the acting force of the ejection key 21, then moves along the energy storage channel 12 and drives the traction elastic arm 22 and the leaf spring 23 to store energy, and when the first motion control part 25 crosses and disengages from the energy storage channel 12, the first motion control part 25 drives the base body 24 to move under the traction force of the traction elastic arm 22 and the reaction force of the energy storage channel 12;

The second motion control part 241 and the third motion control part 242 are assembled on the emission channel 13, and the base body 24 is a component connected with the cutting part 9; when the ejection key 21 is not subjected to an external acting force, the second motion control part 241 and the third motion control part 242 are located in the emission channel 13; when the first motion control part 25 moves along the energy storage channel 12, the movement and rotation tendency of the base body 24 under the elastic action of the leaf spring 23 is blocked by the second motion control part 241 and the third motion control part 242 abutting against the emission channel 13; when the first motion control part 25 crosses and disengages from the energy storage channel 12, the second motion control part 241 and the third motion control part 242 move along the emission channel 13 toward the lancing opening 101 driven by the base body 24; after the second motion control part 241 crosses and disengages from the emission channel 13, the base body 24 moves to one lower side relative to the lancing opening 101 under the torsion force of the leaf spring 23 and the inertial acting force along the emission channel 13 in the early stage, and accordingly the base body 24 drives the cutting part 9 to perform lancing toward the other lower side.

Through the application of Embodiment 1 of the present disclosure, the integrated ejection mechanism component 2 has a pre-emission state, an initial emission state, a late emission state and a final emission state, and is converted into the four states in sequence. The conversion process of the four states is described in detail as follows:

In the pre-emission state, the integrated ejection mechanism component 2 is placed in the housing 1 in a natural unstressed state, the ejection key 21 is mounted on the ejection channel 11, the first motion control part 25 is located at the placement end 121 of the energy storage channel 12, and the second motion control part 241 and the third motion control part 242 are located in the emission channel 13 and close to the two sides of the inner wall of the emission channel 13 respectively.

When converting from the pre-emission state to the initial emission state, the ejection key 21 pushes the first motion control part 25 to move along the energy storage channel 12 under an external force, the traction elastic arm 22 and the leaf spring 23 move and store energy synchronously, and the movement and rotation tendency of the base body 24 under the elastic action of the leaf spring 23 is blocked by the two sides of the inner wall of the emission channel 13.

When converting from the initial emission state to the late emission state, the ejection key 21 continues to push the first motion control part 25 to move along the energy storage channel 12 under the external force, the traction elastic arm 22 and the leaf spring 23 continue to move and store energy synchronously, wherein the first motion control part 25 moves to the critical end 122 of the energy storage channel 12 and is in a critical to-be-disengaged state.

When converting from the late emission state to the final emission state, the ejection key 21 continues to push the first motion control part 25 under the external force to make the first motion control part 25 cross the critical end 122 of the energy storage channel 12, the first motion control part 25 drives the base body 24 to move along the emission channel 13 toward the lancing opening 101 under the traction force of the traction elastic arm 22 and the reaction force of the energy storage channel 12, the second motion control part 241 on the base body 24 disengages from the emission channel 13 to release the restriction on the rotation tendency of the base body 24, and the base body 24 and the cutting part 9 on the base body 24 perform rotary lancing at the lancing opening 101 under the torsion force of the leaf spring 23.

Through the implementation of the above embodiment of the present disclosure, in the pre-emission state, the integrated ejection mechanism component 2 is placed in the housing 1 in a natural unstressed state, and the leaf spring 23 of the integrated ejection mechanism component 2 that provides elastic acting force for the rotation of the base body 24 is not stressed, which can well avoid the possibility of product failure before use, and ensure that sufficient and rapid lancing force can be provided for the lancing of the cutting part 9 in the final emission state, so as to guarantee the blood collection volume and reduce pain;

Through the implementation of the above embodiment of the present disclosure, when the first motion control part 25 bears the acting force of the ejection key 21, it moves along the energy storage channel 12 and drives the traction elastic arm 22 and the leaf spring 23 to store energy, the movement and rotation tendency of the base body 24 under the elastic action of the leaf spring 23 is blocked first, so that the traction elastic arm 22 and the leaf spring 23 can generate sufficient deformation to store enough elastic acting force; and when the first motion control part 25 crosses and disengages from the energy storage channel 12, the first motion control part 25 drives the base body 24 to move under the traction force of the traction elastic arm 22 and the reaction force of the energy storage channel 12, at this time, the traction elastic arm 22 can provide sufficient elastic acting force for the movement of the base body 24, so that the base body 24 can move rapidly along the emission channel 13 toward the lancing opening 101 with sufficient emission actuating and high emission speed.

Through the implementation of the above embodiment of the present disclosure, after the second motion control part on the base body 24 is driven by the rapid traction of the traction elastic arm 22 to cross and disengage from the emission channel 13, the base body 24 immediately rotates under the action of the leaf spring 23 after the rotation restriction is released, and the leaf spring 23 which has stored sufficient elastic acting force in the early stage can drive the cutting part 9 on the base body 24 to perform rapid rotary lancing with large and high-speed lancing force, thus ensuring the blood collection volume and reducing pain.

In another embodiment of the present disclosure, the first motion control part 25, the second motion control part 241 and the third motion control part 242 are all raised cylindrical bodies, and the ejection channel 11, the energy storage channel 12 and the emission channel 13 are all grooves or spaces arranged on the housing 1. This design makes the structural design of the integrated ejection mechanism component 2, the movement of each component and the realization of the elastic structures of the traction elastic arm 22 and the leaf spring 23 more scientific and reasonable, and makes the whole pushing and emission process of the lancet smoother without jamming.

In another embodiment thereof, a first retaining wall 131 and a second retaining wall 132 are arranged on the housing 1, the space between the first retaining wall 131 and the second retaining wall 132 forms the emission channel 13, a disengagement end 1311 is arranged on the first retaining wall 131 at a position close to the lancing opening 101, and an abutment end 1321 is arranged on the second retaining wall 132 at a position close to the lancing opening 101;

In the pre-emission state, the second motion control part 241 is located in the emission channel 13 and close to the first retaining wall 131, and the third motion control part 242 is located in the emission channel 13 and close to the second retaining wall 132;

When converting from the pre-emission state to the initial emission state, the movement and rotation tendency of the base body 24 under the elastic action of the leaf spring 23 is blocked by the first retaining wall 131 and the second retaining wall 132;

When converting from the late emission state to the final emission state, the second motion control part 241 on the base body 24 moves and disengages from the first retaining wall 131 to release the restriction on the rotation tendency of the base body 24, and the third motion control part 242 moves to the abutment end 1321 and is rotatably connected with the abutment end 1321 in a matching manner.

In another embodiment, with the end face of the housing 1 where the lancing opening 101 is located as a horizontal reference plane, the horizontal height of one of the second motion control part 241 and the third motion control part 242 is higher than that of the other, thus forming a height difference between the second motion control part 241 and the third motion control part 242. When converting from the pre-emission state to the initial emission state, this height difference can make the two parts better blocked by the emission channel 13 (the first retaining wall 131 and the second retaining wall 132) without premature disengagement; and when converting from the late emission state to the final emission state, when one part is released from the restriction of the emission channel 13, it can better rotate around the other part under the action of the leaf spring 23 with a sufficiently good lancing angle and speed.

In another embodiment, a driving surface 210 is arranged on the ejection key 21, and a driven part 251 is arranged on the first motion control part 25 or at the position between the traction elastic arm 22 and the leaf spring 23 corresponding to the driving surface 210; when the ejection key 21 bears an external acting force, the driving surface 210 contacts and acts on the driven part 251, so that the ejection key 21 drives and pushes the first motion control part 25 to move.

Specifically, the driving surface 210 is arranged to be an inclined surface 2101 at least at the part in contact with the driven part 251, and the driven part 251 is arranged to be a curved surface 2511 at least at the part in contact with the driving surface 210, so that the contact part of the driving surface 210 and the driven part 251 on the cross section is a point contact. Thus, when pushing the ejection key 21 to drive the first motion control part 25 to move, especially when the energy storage channel 12 is not a straight channel, the driving surface 210 and the driven part 251 can always keep point contact when the driven part 251 rotates at a certain angle, thereby reducing friction force, making the pushing process smoother, reducing the force applied to push the ejection key 21 and avoiding jamming.

In Embodiment 1 of the present disclosure, the traction elastic arm 22 is curved, a curved transition part 221 is arranged on the traction elastic arm 22 at the connection part with the first motion control part 25, and the traction elastic arm 22 and the transition part 221 are curved in different directions. Specifically, the main body of the traction elastic arm 22 and the transition part 221 are reverse circular arcs, so that when pushing the ejection key 21 and the first motion control part 25 pushes the traction elastic arm 22 and the leaf spring 23 to store energy, the resistance generated after the compression of the traction elastic arm 22 will diverge and not concentrate on a single point; and when the first motion control part 25 disengages from the energy storage channel 12 so that the traction elastic arm 22 provides traction force for the movement of the base body 24 toward the lancing opening 101, the tension of the traction elastic arm 22 stretches uniformly without being too large or too small, resulting in a better hand feeling.

Furthermore, the energy storage channel 12 is curved, and the bending direction of the energy storage channel 12 is set toward the lancing opening 101, so that the middle position of the energy storage channel 12 is higher than the two ends. The first motion control part 25 is first pushed obliquely upward from the placement end of the energy storage channel 12 to the middle, and then pushed obliquely downward from the middle to the critical end 122, which makes the energy storage and release process of the traction elastic arm 22 and the leaf spring 23 more reasonable and the operation hand feeling better.

Specifically, the emission channel 13 is obliquely arranged toward the lancing opening 101, specifically inclined in the general direction toward the lancing opening 101, so that the base body 24 can move obliquely downward better under the traction of the traction elastic arm 22, and the process of the disengagement of the second motion control part 241 and the abutment of the third motion control part to the abutment end 1321 can be smoother.

In another embodiment of the present disclosure, the ejection key 21 is provided with an ejection part 212 for bearing an external pressing force and/or a push-pull force; an ejection opening 102 is arranged on the housing 1 at a position corresponding to the ejection part 212;

When the ejection part 212 is located at the head of the ejection key 21, the ejection opening 102 is arranged on the peripheral side of the housing 1;

When the ejection part 212 is located on the two sides of the ejection key 21, the ejection opening 102 is arranged on the two sides of the housing 1;

When the ejection part 212 is located at both the head and the two sides of the ejection key 21, the ejection openings 102 are arranged on both the peripheral side and the two sides of the housing 1.

To better understand the composition and function of each component in the present disclosure, the specific composition and use steps of the present disclosure will be described in detail below in combination with a detailed embodiment.

The blood collection ejection actuating mechanism proposed in this detailed embodiment comprises an integrated ejection mechanism component 2, and an ejection channel 11, an energy storage channel 12 and an emission channel 13 which are arranged on the housing 1 for the assembly and movement of the integrated ejection mechanism component 2.

In this detailed embodiment, the structure of the housing 1 is referred to FIG. 11: the ejection channel 11, the energy storage channel 12 and the emission channel 13 are arranged on the housing 1, and all of them are grooves or spaces on the housing 1; one end of the energy storage channel 12 is a placement end 121 and the other end is a critical end 122, the energy storage channel 12 is curved and its bending direction is set toward the lancing opening 101; the first retaining wall 131 and the second retaining wall 132 are arranged on the housing 1, the space between them forms the emission channel 13, the disengagement end 1311 is arranged on the first retaining wall 131 close to the lancing opening 101, the abutment end 1321 is arranged on the second retaining wall 132 close to the lancing opening 101, and the emission channel 13 is obliquely arranged toward the lancing opening 101; the housing 1 is also provided with the lancing opening 101 for the cutting part 9 to extend out, and the ejection opening 102 on the other peripheral side of the lancing opening 101 for the movement of the ejection key 21.

In this detailed embodiment, the integrated ejection mechanism component 2 includes an ejection key 21, a traction elastic arm 22, a leaf spring 23 and a base body 24 which are integrally and continuously arranged in sequence; a first motion control part 25 is arranged between the traction elastic arm 22 and the leaf spring 23; a second motion control part 241 and a third motion control part 242 are arranged on the base body 24, wherein the second motion control part 241 is far away from the lancing opening 101 and the third motion control part 242 is close to the lancing opening 101; the first motion control part 25, the second motion control part 241 and the third motion control part 242 are all raised cylindrical bodies. The ejection key 21 is provided with a guide part 211 assembled on the ejection channel 11 and an ejection part 212 for bearing an external pressing force and/or a push-pull force; the driving surface 210 is arranged on the ejection key 21, and the driven part 251 is arranged on the first motion control part 25 or at the position between the traction elastic arm 22 and the leaf spring 23 corresponding to the driving surface 210; when the ejection key 21 bears an external acting force, the driving surface 210 contacts and acts on the driven part 251, so that the ejection key 21 drives and pushes the first motion control part 25 to move. The driving surface 210 is an inclined surface 2101 at least at the part in contact with the driven part 251, and the driven part 251 is a curved surface 2511 at least at the part in contact with the driving surface 210, so that the contact part of the two on the cross section is a point contact. The traction elastic arm 22 is curved, a curved transition part 221 is arranged on the traction elastic arm 22 at the connection part with the first motion control part 25, and the traction elastic arm 22 and the transition part 221 are curved in different directions.

The blood collection ejection actuating mechanism in this detailed embodiment is configured to have a pre-emission state, an initial emission state, a late emission state and a final emission state, and can be sequentially converted from the pre-emission state to the initial emission state, then to the late emission state, and finally to the final emission state.

In the pre-emission state, the ejection key 21 and the first motion control part 25 are respectively placed in the corresponding channels of the lower shell, and the second motion control part 241 and the third motion control part 242 are respectively placed close to the corresponding retaining walls.

In the initial emission state, the inclined surface 2101 of the ejection key 21 is subjected to a force F to push the first motion control part 25 to move leftward, and the leaf spring 23 and the traction elastic arm 22 move leftward synchronously and store energy. The counterclockwise rotation tendency of the base body 24 under the elastic force of the leaf spring 23 is blocked by the first retaining wall 131 and the second retaining wall 132.

In the late emission state, the inclined surface 2101 of the ejection key 21 is continuously subjected to the force F to push the first motion control part 25 to move leftward, and the leaf spring 23 and the traction elastic arm 22 continue to move leftward synchronously and store energy, wherein the first motion control part 25 moves to the end of the energy storage channel 12 and is in a critical to-be-disengaged state.

In the final emission state, the inclined surface 2101 of the ejection key 21 is further subjected to the force F to push the first motion control part 25 to move leftward across the critical end 122; the first motion control part 25 starts to move toward the lower opening under the traction force of the traction elastic arm 22 and the reaction force of the energy storage channel 12. Since the base body 24 is integrated with the first motion control part 25, it also moves along the emission channel 13 which is obliquely downward to the left formed by the first retaining wall 131 and the second retaining wall 132. After the second motion control part 241 disengages from the first retaining wall 131, the base body 24 moves downward to the left relative to the lancing opening 101 under the torsion force of the leaf spring 23 and the inertial acting force along the emission channel 13 in the early stage, and accordingly the base body 24 drives the cutting part 9 to perform lancing downward and to the right.

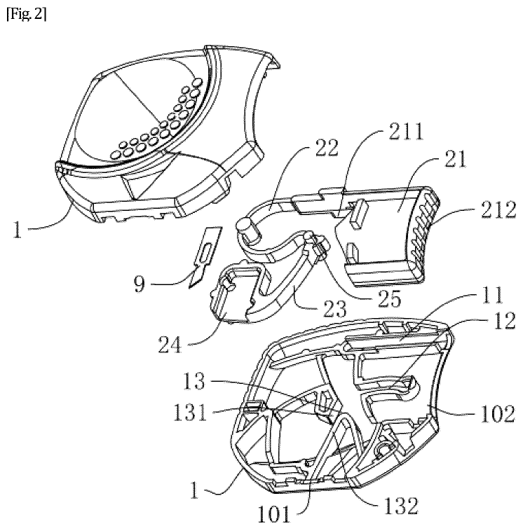

Thus, under the action of the integrated ejection actuating mechanism, the base body 24 can realize the function of moving downward first and then rotating, thereby driving the cutting part 9 to perform lancing. As shown in FIG. 2, the cutting part 9 is a blade, and the lancing is realized by the blade cutting through the skin.

Embodiment 2: Embodiment 2 of the present disclosure discloses a lancet which uses the blood collection ejection actuating mechanism described in Embodiment 1. The lancet adopts a lancing (cutting) blood collection mode, which is mainly applicable to heel blood collection of infants and young children, and can also be applied to blood collection at other parts of the human body.

The possible variations of the present disclosure for the above embodiments are described as follows:
1. In the above embodiments, the second motion control part 241 is arranged at a higher horizontal height than the third motion control part 242, and the base body 24 rotates around the third motion control part 242 after the second motion control part 241 disengages from the emission channel 13; the present disclosure is not limited to this, and the second motion control part 241 can also be arranged at a lower horizontal height than the third motion control part 242, with the base body 24 still rotating around the third motion control part 242 after the disengagement of the second motion control part 241.
2. In the above embodiments, the housing 1 comprises an upper shell and a lower shell, and one or more of the ejection channel 11, the energy storage channel 12 and the emission channel 13 are arranged on either or both of the upper shell and the lower shell. For example, as shown in FIG. 11, the ejection channel 11, the energy storage channel 12 and the emission channel 13 can all be arranged on the lower shell; since one side of the base body 24 needs to be equipped with the cutting part 9 as shown in the figure, the emission channel 13 only needs to be arranged on the lower shell, and alternatively, the emission channel 13 is arranged on the lower shell while the ejection channel 11 and the energy storage channel 12 are arranged on both the upper shell and the lower shell.
3. In the above embodiments, the ejection key 21 is provided with the ejection part 212 for bearing an external pressing force and/or a push-pull force, and the ejection opening 102 is arranged on the housing 1 at a position corresponding to the ejection part 212. The positions of the ejection part 212 and the ejection opening 102 are not limited to the structure shown in FIG. 11: when the ejection part 212 is located at the head of the ejection key 21, the ejection opening 102 is arranged on the peripheral side of the housing 1; when the ejection part 212 is located on the two sides of the ejection key 21, the ejection opening 102 is arranged on the two sides of the housing 1; when the ejection part 212 is located at both the head and the two sides of the ejection key 21, the ejection opening 102 is arranged on both the peripheral side and the two sides of the housing 1.
4. In the above embodiments, the curved transition part 221 is arranged on the traction elastic arm 22 at the connection part with the first motion control part 25; the present disclosure is not limited to this, and the transition part 221 can also be in the shape of a rectangle or a square.
5. In the above embodiments, the lancing opening 101 can be an open opening, a part that is closed before use and opened only when in use, or a position where a film is arranged at the extending position of the cutting part 9, so that the cutting part 9 first cuts through the film when extending out from the lancing opening 101 and then cuts the blood collection site.
6. In the above embodiments, the cutting part 9 is a blade, but the present disclosure is not limited thereto; the cutting part 9 may also be a needle or other sharp objects capable of lancing human skin.
7. In the above embodiments, the cutting part 9 is fixedly connected to the base body by means of assembly, and alternatively, the cutting part 9 may be integrally formed with the base body 24. As long as the cutting part 9 has a certain hardness and sharpness, blood collection can also be achieved effectively.

The above embodiments are merely intended to illustrate the technical conception and characteristics of the present disclosure, and their purpose is to enable those skilled in the art to understand the content of the present disclosure and implement it accordingly, and shall not be construed as limiting the protection scope of the present disclosure. All equivalent changes or modifications made in accordance with the spirit and essence of the present disclosure shall fall within the protection scope of the present disclosure.

1. A blood collection ejection actuating mechanism for providing ejection actuating for the emission and lancing of a cutting part of a lancet, the lancet comprising a housing with a lancing opening for the cutting part to extend out;
the blood collection ejection actuating mechanism comprises an integrated ejection mechanism component, and an ejection channel, an energy storage channel and an emission channel which are arranged on the housing for the assembly and movement of the integrated ejection mechanism component;
the integrated ejection mechanism component comprises an ejection key, a traction elastic arm a leaf spring and a base body which are integrally and continuously arranged in sequence; a first motion control part is arranged between the traction elastic arm, and the leaf spring; a second motion control part and a third motion control part are arranged on the base body;
the ejection key is provided with a guide part assembled on the ejection channel, and the ejection key is a component that bears an external pressing force and/or a push-pull force, then moves along the ejection channel and pushes the first motion control part to move; a driving surface is arranged on the ejection key;
the first motion control part is assembled on the energy storage channel, and a driven part is arranged on the first motion control part or at the position between the traction elastic arm and the leaf spring corresponding to the driving surface; the first motion control part is a component that bears the acting force of the ejection key, then moves along the energy storage channel and drives the traction elastic arm and the leaf spring to store energy, and when the first motion control part crosses and disengages from the energy storage channel, the first motion control part drives the base body to move under the traction force of the traction elastic arm and the reaction force of the energy storage channel;
the second motion control part and the third motion control part are assembled on the emission channel, and the base body is a component connected with the cutting part; when the ejection key is not subjected to an external acting force, the second motion control part and the third motion control part are located in the emission channel; when the first motion control part moves along the energy storage channel, the tendency of the base body to move and rotate under the elastic action of the leaf spring is blocked by the second motion control part and the third motion control part abutting against the emission channel; when the first motion control part crosses and disengages from the energy storage channel, the second motion control part and the third motion control part are driven by the base body to move along the emission channel toward the lancing opening; after the second motion control part crosses and disengages from the emission channel, the base body moves to one lower side relative to the lancing opening under the torsion force of the leaf spring and the inertial acting force along the emission channel in the early stage, and accordingly the base body drives the cutting part to perform lancing toward the other lower side.

2. The blood collection ejection actuating mechanism according to claim 1, wherein the blood collection ejection actuating mechanism is configured to be sequentially converted into a pre-emission state, an initial emission state, a late emission state and a final emission state in order;
in the pre-emission state, the integrated ejection mechanism component is placed in the housing in a natural unstressed state, the ejection key is mounted on the ejection channel, the first motion control part is located at the placement end of the energy storage channel and the second motion control part and the third motion control part, are located in the emission channel and close to the two sides of the inner wall of the emission channel respectively;
when converting from the pre-emission state to the initial emission state, the ejection key pushes the first motion control part to move along the energy storage channel under an external force, the traction elastic arm and the leaf spring move and store energy synchronously, and the tendency of the base body to move and rotate under the elastic action of the leaf spring is blocked by the two sides of the inner wall of the emission channel;
when converting from the initial emission state to the late emission state, the ejection key continues to push the first motion control part to move along the energy storage channel under the external force, the traction elastic arm and the leaf spring continue to move and store energy synchronously, wherein the first motion control part moves to the critical end of the energy storage channel and is in a critical to-be-disengaged state;
when converting from the late emission state to the final emission state, the ejection key continues to push the first motion control part under the external force to make the first motion control part cross the critical end of the energy storage channel, the first motion control part drives the base body to move along the emission channel toward the lancing opening under the traction force of the traction elastic arm and the reaction force of the energy storage channel; after the second motion control part on the base body disengages from the emission channel, the restriction on the rotation tendency of the base body is released, and the base body moves to one lower side relative to the lancing opening under the torsion force of the leaf spring and the inertial acting force along the emission channel in the early stage, and accordingly the base body drives the cutting part to perform lancing toward the other lower side.

3. The blood collection ejection actuating mechanism according to claim 2, wherein: the first motion control part, the second motion control part and the third motion control part are all raised cylindrical bodies, and the ejection channel, the energy storage channel and the emission channel are all grooves or spaces arranged on the housing.

4. The blood collection ejection actuating mechanism according to claim 3, wherein:
a first retaining wall and a second retaining wall are arranged on the housing, the space between the first retaining wall and the second retaining wall forms the emission channel a disengagement end is arranged on the first retaining wall at a position close to the lancing opening, and an abutment end is arranged on the second retaining wall at a position close to the lancing opening;
in the pre-emission state, the second cylindrical body is located in the emission channel and close to the first retaining wall, and the third cylindrical body is located in the emission channel and close to the second retaining wall; when converting from the pre-emission state to the initial emission state, the tendency of the base body to move and rotate under the elastic action of the leaf spring is blocked by the first retaining wall and the second retaining wall;
when converting from the late emission state to the final emission state, after the second motion control part on the base body moves and disengages from the first retaining wall, the restriction on the rotation tendency of the base body is released; after the third motion control part moves to the abutment end, it is rotatably matched and connected with the abutment end.

5. The blood collection ejection actuating mechanism according to claim 1, wherein: with the end face of the housing where the lancing opening is located as a horizontal reference plane, the horizontal height of one of the second motion control part and the third motion control part is higher than that of the other.

6. The blood collection ejection actuating mechanism according to claim 1, wherein: the driving surface is arranged to be an inclined surface at least at the part in contact with the driven part, and the driven part is arranged to be a curved surface at least at the part in contact with the driving surface, so that the contact part of the driving surface and the driven part on the cross section is a point contact.

7. The blood collection ejection actuating mechanism according to claim 1, wherein the traction elastic arm is curved, a curved transition part is arranged on the traction elastic arm at the connection part with the first motion control part, and the traction elastic arm and the transition part are curved in different directions.

8. The blood collection ejection actuating mechanism according to claim 1, wherein:
the ejection key is provided with an ejection part for bearing an external pressing force and/or a push-pull force;
an ejection opening is arranged on the housing at a position corresponding to the ejection part;
when the ejection part is located at the head of the ejection key, the ejection opening is arranged on the peripheral side of the housing;
when the ejection part is located on the two sides of the ejection key, the ejection opening is arranged on the two sides of the housing;
when the ejection part is located at both the head and the two sides of the ejection key, the ejection opening is arranged on both the peripheral side and the two sides of the housing.

9. The blood collection ejection actuating mechanism according to claim 1, wherein: the emission channel is obliquely arranged toward the lancing opening.

10. The blood collection ejection actuating mechanism according to claim 2, wherein: the energy storage channel is curved, and the bending direction of the energy storage channel is set toward the lancing opening.

11. A lancet, wherein: the lancet adopts the blood collection ejection actuating mechanism according to claim 1.

12. The lancet according to claim 11, wherein: the housing comprises an upper shell and a lower shell, and the ejection channel, the energy storage channel and the emission channel are arranged on either or both of the upper shell and the lower shell.
